SEQUENCE LISTING (38 sequences)

SEQ ID NO: 1 - DNA, 54 bp, Artificial Sequence
  DNA sequence encoding transacting ribozyme
agatctaaaaaaaaacctgatgagtccgtgaggacgaaactggttaaaagatct

SEQ ID NO: 2 - DNA, 54 bp, Artificial Sequence
  DNA sequence encoding transacting ribozyme
agatctaaattatccactgatgagtccgtgaggacgaaacgggcgaaaagatct

SEQ ID NO: 3 - DNA, 54 bp, Artificial Sequence
  DNA sequence encoding transacting ribozyme
agatctaaatcgttacctgatgagtccgtgaggacgaaactaccgaaaagatct

SEQ ID NO: 4 - DNA, 54 bp, Artificial Sequence
  DNA sequence encoding transacting ribozyme
agatctaaatgatgttctgatgagtccgtgaggacgaaaccacttaaaagatct

SEQ ID NO: 5 - DNA, 54 bp, Artificial Sequence
  DNA sequence encoding transacting ribozyme
agatctaaattttccactgatgagtccgtgaggacgaaacgtgcaaaaagatct

SEQ ID NO: 6 - DNA, 56 bp, Artificial Sequence
  DNA sequence encoding transacting ribozyme
agatctaaattgataccctgatgagtccgtgaggacgaaacagtcagaaaagatct

SEQ ID NO: 7 - DNA, 54 bp, Artificial Sequence
  DNA sequence encoding transacting ribozyme
agatctaaattcgtttctgatgagtccgtgaggacgaaacaccacaaaagatct

SEQ ID NO: 8 - DNA, 165 bp, Artificial Sequence
  DNA sequence encoding target ribozyme
agatctaaacgccgatctgatgagtccgtgaggacgaaactttaaaaaccaaggagatctaaacatctcactgatgagtccgtgaggacgaaacattacgaaaccaaggagatctaaatcattcacctgatgagtccgtgaggacgaaactttagcaaaccaagg

SEQ ID NO: 9 - DNA, 162 bp, Artificial Sequence
  DNA sequence encoding target ribozyme
agatctaaacgttagtctgatgagtccgtgaggacgaaaccaacaaaaccaaggagatctaaaggcatcactgatgagtccgtgaggacgaaactgttaaaaccaaggagatctaaaccacatcctgatgagtccgtgaggacgaaacagtttaaaccaagg

SEQ ID NO: 10 - DNA, 162 bp, Artificial Sequence; identical to SEQ ID NO: 9
  DNA sequence encoding target ribozyme

SEQ ID NO: 11 - DNA, 162 bp, Artificial Sequence
  DNA sequence encoding target ribozyme
agatctaaaagagcgctgatgagtccgtgaggacgaaacagtcaaaaccaaggagatctaaatttcgatctgatgagtccgtgaggacgaaaccagctaaaccaaggagatctaaacgatttcctgatgagtccgtgaggacgaaacatcaccaaaccaagg

SEQ ID NO: 12 - DNA, 56 bp, Artificial Sequence
  DNA sequence encoding target ribozyme
agatctaaatgcgtctgatgagtccgtgaggacgaaacaggcaggtaaaaccaagg

SEQ ID NO: 13 - DNA, 157 bp, Artificial Sequence
  DNA sequence encoding target ribozyme
agatctaaactcgtcctgatgagtccgtgaggacgaaacgatcaaaaccaaggagatctaaagggcgctgatgagtccgtgaggacgaaacgcgaaaaccaaggagatctaaagtactcctgatgagtccgtgaggacgaaaccagcgaaaccaagg

SEQ ID NO: 14 - DNA, 168 bp, Artificial Sequence
  DNA sequence encoding target ribozyme
agatctaaaactaaatgctgatgagtccgtgaggacgaaacgagttaaaaccaaggagatctaaagtttaataactgatgagtccgtgaggacgaaacttgttcaaaccaaggagatctaaaacttttgctgatgagtccgtgaggacgaaacgtgtataaaccaagg

SEQ ID NO: 15 - DNA, 162 bp, Artificial Sequence
  DNA sequence encoding target ribozyme
agatctaaaggtccatctgatgagtccgtgaggacgaaacaaagcaaaccaaggagatctaaacaggttcctgatgagtccgtgaggacgaaacaatgtaaaccaaggagatctaaatcgctttctgatgagtccgtgaggacgaaacgtgataaaccaagg

SEQ ID NO: 16 - DNA, 160 bp, Artificial Sequence
  DNA sequence encoding target ribozyme
agatctaaagctcgatctgatgagtccgtgaggacgaaacgaaccaaaccaaggagatctaaacgagtcctgatgagtccgtgaggacgaaaccgggaaaccaaggagatctaaagtcgatgctgatgagtccgtgaggacgaaacttcgcaaaccaagg

SEQ ID NO: 17 - DNA, 56 bp, Artificial Sequence
  DNA sequence encoding target ribozyme
agatctaaatgcgtctgatgagtccgtgaggacgaaacaggcaggcgaaaccaagg

SEQ ID NO: 18 - DNA, 38 bp, Artificial Sequence
  DNA sequence encoding target ribozyme
tgctcttctgatgagtccgtgaggacgaaaccgcctga

SEQ ID NO: 19 - DNA, 39 bp, Artificial Sequence
  DNA sequence encoding target ribozyme
ttcaaagactgatgagtccgtgaggacgaaacgaggatc

SEQ ID NO: 20 - DNA, 34 bp, Artificial Sequence
  DNA sequence encoding target ribozyme
gtccatctgatgagtccgtgaggacgaaaccggc

SEQ ID NO: 21 - DNA, 36 bp, Artificial Sequence
  DNA sequence encoding target ribozyme
attagagctgatgagtccgtgaggacgaaacaaacg

SEQ ID NO: 22 - DNA, 37 bp, Artificial Sequence
  DNA sequence encoding target ribozyme
gtcctgactgatgagtccgtgaggacgaaacattgca

SEQ ID NO: 23 - DNA, 43 bp, Artificial Sequence
  DNA sequence encoding target ribozyme
tcgttgtctctgatgagtccgtgaggacgaaacatgacaccga

SEQ ID NO: 24 - DNA, 39 bp, Artificial Sequence
  DNA sequence encoding target ribozyme
gcgaggagctgatgagtccgtgaggacgaaacatggtgt

SEQ ID NO: 25 - DNA, 37 bp, Artificial Sequence
  DNA sequence encoding target ribozyme
aacttttctgatgagtccgtgaggacgaaacataatg

SEQ ID NO: 26 - DNA, 42 bp, Artificial Sequence
  DNA sequence encoding target ribozyme
tcgaagctgtctgatgagtccgtgaggacgaaaccgcgttga

SEQ ID NO: 27 - DNA, 37 bp, Artificial Sequence
  DNA sequence encoding target ribozyme
atcagggtctgatgagtccgtgaggacgaaaggtgcc

SEQ ID NO: 28 - DNA, 37 bp, Artificial Sequence
  DNA sequence encoding target ribozyme
tcttcgactgatgagtccgtgaggacgaaacatggct

SEQ ID NO: 29 - DNA, 37 bp, Artificial Sequence
  DNA sequence encoding target ribozyme
tagcacactgatgagtccgtgaggacgaaacgtttga

SEQ ID NO: 30 - DNA, 36 bp, Artificial Sequence
  DNA sequence encoding target ribozyme
tgcaatactgatgagtccgtgaggacgaaactgcct

SEQ ID NO: 31 - DNA, 36 bp, Artificial Sequence
  DNA sequence encoding target ribozyme
aagtcatctgatgagtccgtgaggacgaaacctgga

SEQ ID NO: 32 - DNA, 36 bp, Artificial Sequence
  DNA sequence encoding target ribozyme
gataaggctgatgagtccgtgaggacgaaactttcc

SEQ ID NO: 33 - DNA, 36 bp, Artificial Sequence
  DNA sequence encoding target ribozyme
catattcctgatgagtccgtgaggacgaaacactcg

SEQ ID NO: 34 - DNA, 38 bp, Artificial Sequence
  DNA sequence encoding target ribozyme
tcatgtatctgatgagtccgtgaggacgaaacaaaagg

SEQ ID NO: 35 - DNA, 36 bp, Artificial Sequence
  DNA sequence encoding target ribozyme
ggttaaactgatgagtccgtgaggacgaaacttggg

SEQ ID NO: 36 - DNA, 36 bp, Artificial Sequence
  DNA sequence encoding target ribozyme
gtccagtctgatgagtccgtgaggacgaaacttaag

SEQ ID NO: 37 - DNA, 139 bp, Artificial Sequence
  DNA sequence encoding target ribozyme
gcggccgctcgagctctgatgagtccgtgaggacgaaacggtacccggtaccgtcagctcgagctcagatctggatccgtcgacggatctagatcgtcctgatgagtccgtgaggacgaaacggatctgcagcggccgc

SEQ ID NO: 38 - DNA, 176 bp, Artificial Sequence
  DNA sequence encoding target ribozyme
gcggccgctcgagctctgatgagtccgtgaggacgaaacggtacccggtaccgtcagctcgagctctnnnnnnctgatgagtccgtgaggacgaaannnnnnagatctggatccgtcgacggatctagatccgtcctgatgagtccgtgaggacgaaacggatctgcagcggccgc

(19) United States
(12) Patent Application Publication
(10) Pub. No.: US 2003/0125280 A1
(43) Pub. Date: Jul. 3, 2003

(54) Tissue specific and target RNA-specific ribozymes as antimicrobial therapeutics against microbial pathogens

(75) Inventors: James S. Norris, Mt. Pleasant, SC (US); Michael G. Schmidt, Mt. Pleasant, SC (US); Joseph W. Dolan, Mt. Pleasant, SC (US); Steven D. London, Mt. Pleasant, SC (US); Harold D. May, Mt. Pleasant, SC (US); Gary A. Clawson, Bethesda, MD (US)

Correspondence Address: Janis K. Fraser, Ph.D., J.D., Fish & Richardson, P.C., 225 Franklin Street, Boston, MA, 02110-2804, US

(73) Assignees: Medical University of South Carolina; The Penn State Research Foundation, a Pennsylvania corporation

(21) Appl. No.: 10/127,025

(22) Filed: Apr. 19, 2002

Related U.S. Application Data
(63) Continuation of application No. 09/319,395, filed on Oct. 5, 1999, now abandoned.
Application No. 09/319,395 is a 371 of international application No. PCT/US97/21922, filed on Dec. 2, 1997.
(60) Provisional application No. 60/032,261, filed on Dec. 3, 1996.

(51) Int. Cl. A61K 48/00; A61K 9/127; C12N 15/86; C12N 15/88; C12N 7/00

(52) U.S. Cl. 514/44; 424/93.2; 424/450; 435/235.1; 435/456; 435/458

(57) ABSTRACT

The invention provides compositions comprising the tissue specific and target RNA-specific ribozyme(s) in either a viral delivery system or a biologic liposome preparation, wherein the viral delivery system or a biologic liposome comprises a pathogen-specific promotor upstream from a sequence encoding a triple ribozyme comprising a) a 5′ autocatalytically cleaving ribozyme sequence, b) a catalytic ribozyme comprising a target RNA-specific binding site and c) a 3′ autocatalytically cleaving ribozyme sequence. The invention also provides methods of treating and/or preventing bacterial infections by administering the compositions of the invention.

### BACKGROUND

1. Field of the Invention

The invention relates to the delivery of ribozymes to microorganisms in order to treat infections. More specifically, the invention relates to the use of virions to package and deliver DNA encoding ribozymes to the an infected patient. Most specifically, the invention relates to the use of target specific virions to package and deliver DNA comprising a target specific promoter and encoding a ribozyme directed to the target organisms nucleic acids.

2. Background Art

Infectious diseases sicken or kill millions of people each year. Numerous antimicrobial therapies have been designed to target one or several infectious agents. These therapies show varying degrees of success in eradicating infection. However, the failure of many of these therapies to target specific infectious agents has lead to overuse or inappropriate use of the therapies, which in turn has lead to the development of drug resistant microbes. The development of drug resistance in many infectious agents has reduced the efficacy and increased the risk of using the traditional antimicrobial therapies.

While ribozymes have been known and studied for several years, they have not been used in the treatment of bacterial infections. There are many reasons for this. A key technical concern in the use of ribozymes as antimicrobial agents is that the ribozyme must be taken up and expressed by the targeted microbe so that the ribozyme(s) can cleave the targeted RNA(s) inside the microorganism. This concern is addressed by the present invention.

### SUMMARY OF THE INVENTION

The present invention, RiboZide™, is a microbiocidal agent directed against any viral, bacterial, fungal, or other single or multicellular organism from any known taxonomic family, genus, or species, and from previously unknown, or uncharacterized organism. The present composition of matter has resulted from the development of a new process that delivers a series of ribozymes directed against fundamental and essential cellular processes specific to a targeted microorganism through an inactivated, altered, virus (virion), or abiologic delivery vehicles, capable of delivering a nucleic acid containing the ribozyme(s) into the targeted microorganism. The microorganisms maybe any virus, nonvirus, bacterium, or lower eukaryotes such as fungi, yeast, parasites, protozoa, or other eukaryotes that may be consider normal flora or pathogens of humans, animals, fish, plants or other forms of life.

The present RiboZide™ ribozyme is uniquely suited as an antimicrobial therapeutic in that upon nucleic acid hybridization with the target RNA transcript, the ribozyme-RNA complex achieves a catalytic form that acts as a nuclease to cleave the targeted RNAs. Thus, cleavage deprives the invading microorganism of essential cellular processes which then kill or render it less fit. This approach offers new and unprecedented advances for antimicrobial therapeutics: (1) the preparation bypasses any de novo built-in drug resistance, which sophisticated microbes will be expected to have or develop. (2) cells are generally not capable of counteracting ribozymes delivered into them, (3) microbes have several broad RNA targets that can be attacked in simultaneously with probable synergy, (4) the custom design of the present delivery vehicle can be readily tailored to different families of organisms, (5) the modified delivery vehicle is a non-replicating, artificial construct easy to assemble and manufacture, (6) the preparation can be applied topically or it can be delivered via injection, inhalation, or ingestion, (7) the preparation can be lyophilized and thus confer stability to the antimicrobial therapeutic, (8) the inhalant, ingested or topical form of the antimicrobial therapeutic reduces the immunogenicity of the RiboZide™ preparations as opposed to its parenteral use, and (9) animal test systems exist that enable the evaluation of the RiboZide™ in a measured, incremental fashion to quickly determine the efficacy of the antimicrobial therapeutic agent. Therefore, the combination of the present unique delivery approach and an aggressive mechanism for depriving the microbial cells of essential gene products can achieve the timely defeat of microbes within the host.

The targets of the antimicrobial RiboZide™ therapeutic described herein are the RNAs of invading or normal flora microorganisms. The invention provides the delivery of a series of ribozymes directed towards essential, housekeeping, or virulence genes of one or a series of candidate microorganisms. A ribozyme is uniquely suited as the active component of the present antimicrobial therapeutic in that it is a catalytic RNA molecule that cleaves RNA in a sequence specific manner. Therefore, the catalytically active component of RiboZide™ contains ribozymes that have been designed to inactivate RNA coding for components of the microbial cell. Inactivation of essential proteins and virulence determinants render the invading microbes inactive or slow their growth, while at the same time, the essential processes of the host are not affected.

At the molecular genetic level the coding sequence for a ribozyme or number of ribozymes may be placed under the control of one or more of the following genetic elements: a naturally occurring strong, intermediate or weak constitutively expressed or regulated promoter from the targeted microorganism, or an artificially contrived constitutively expressed or regulated promoter containing either a strong, intermediate or weak consensus sequence that delivers desired levels of ribozyme expression in the targeted microbe. This genetic information is delivered into the microbe by a either a modified virus or abiologic delivery vehicle. This present RiboZide™ is unique in that it contains sufficient genetic information for expression of the ribozyme(s) and such genetic information necessary and sufficient for its assembly and delivery to the targeted microorganism, but does not include nucleic acids native to the virus. Thus, the virion can serve as a molecular vehicle that delivers the inactivating ribozyme(s). Alternatively, an abiologic delivery system (e.g., liposomes) can be used to package nucleic acid carrying the genetic elements necessary and sufficient for the proper expression of the ribozyme(s) into liposomes.

The integration of this fundamental biological information into the present antimicrobial therapeutic (RiboZide™) can be effective for treating plants, fish, animals and humans exposed to microorganisms.

### DESCRIPTION OF THE FIGURES

FIG. 1A shows a schematic of DNA encoding the ribozyme used in the molecular sequence of events in ribozyme maturation and action.

FIG. 1B shows the primary RNA transcript. Autocatalytic cleavage takes place upon completion of transcription.

FIG. 1C shows the release of the trans acting ribozyme. As a direct result of cleavage of the two cis-acting ribozymes, the internal ribozyme containing a reverse and complementary sequence to the mRNA target is released.

FIG. 1D shows the sequence specific hybridization of the ribozyme. The internal or trans acting ribozyme contains a sequence that is reverse and complementary to the targeted message. The ribozyme is synthesized at a concentration sufficient to locate and hybridize to all or substantially all targeted transcripts.

FIG. 1E shows the trans-catalytic cleavage. Upon hybridization of the internal ribozyme to the targeted mRNA transcript, the internal ribozyme achieves a catalytic topology and cleaves the targeted message. Upon cleavage the trans acting ribozyme is released and its activity and function are recycled.

FIG. 2A shows in vitro packaging of the RiboZide™. Purified double-stranded DNA containing a pathogen-specific promoter, ribozymes, transcriptional termination site and packaging elements is mixed with an appropriate concentration of the in vitro packaging extract which contains all of the necessary proteins required for the spontaneous self assembly of viral head around the nucleic acid.

FIG. 2B shows the collection and purification of the intact phage from the components in Panel A.

FIG. 3A shows in vivo packaging of the RiboZide™. Induction of the viral lysogen results in the production the packaging elements that then spontaneously self assembly into a empty virion which are then filled with the ribozyme encoding DNA.

FIG. 3B shows the collection and purification of the intact virion from the components in Panel A.

FIG. 4 schematic representation of the essential genetic elements necessary and sufficient for the production of an active RiboZide™ in a liposome constituting an abiologic delivery vehicle.

FIG. 5 shows the present plasmid, pClip, used to generate the ribozyme encoding DNA construct.

FIG. 6 shows several mutations in the ribozyme that effect catalytic activity.

### DESCRIPTION OF THE INVENTION

As used in the claims, “a” or “an” can mean one or more, depending upon the context in which it is used.

The present invention provides a virion comprising non-viral DNA. The term “virion” as used herein can be a bacteriophage, a mycovirus, or other virus that attacks a bacteria, fungus, yeast, parasite or virus, which can be modified to deliver ribozyme-encoding DNA. The virion can be modified to be non-replicative. Alternatively, if preferred, it can maintain the replicative ability of the native virus.

The virion of the present invention can be any bacteriophage which specifically infects a bacterial pathogen of the present invention as well as any virus which can be specifically targeted to infect the pathogen of the present invention. For example, the bacteriophage can include, but is not limited to, those specific for bacterial cells of the following genera: Bacillus, Campylobacter, Corynebacterium, Enterobacter, Enterococcus, Escherichia, Klebsiella, Mycobacterium, Pseudomonas, Salmonella, Shigella, Staphylococcus, Streptococcus, Vibrio, Streptomyces, Yersinia and the like (see, e.g., the American Type Culture Collection Catalogue of Bacteria and Bacteriophages, latest edition, Rockville, Md.), as well as any other bacteriophages now known or later identified to specifically infect a bacterial pathogen of this invention.

In the present virion, the non-viral DNA can encode a ribozyme. In the present virion, the non-viral DNA can comprises a pathogen-specific promoter upstream from a sequence encoding a triple ribozyme comprising a) a 5′ autocatalytically cleaving ribozyme sequence, b) a catalytic ribozyme comprising a target RNA-specific binding site and c) a 3′ autocatalytically cleaving ribozyme sequence.

The present virion, wherein the non-phage DNA encodes more than one triple ribozyme is also provided. There are several options for constructing the ribozyme encoding sequences: 1) ribozymes directed to different targets in the same pathogen; and 2) multiple copies of the same ribozyme. These be combined in various ways, e.g., multiple copies of DNA encoding 4 different ribozymes in a single construct under one promoter. The promoter can have the chosen level of specificity as described herein.

The virion can contain a nucleic acid encoding at least two different triple ribozymes. The virion can contain a nucleic acid encoding more than one copy of a triple ribozyme. The virion can comprise any ribozyme-encoding nucleic acid, particularly those described herein.

A liposome is provided, comprising a nucleic acid comprising a pathogen-specific promoter upstream from a sequence encoding a triple ribozyme comprising a) a 5′ autocatalytically cleaving ribozyme sequence, b) a catalytic ribozyme comprising a target RNA-specific binding site and c) a 3′ autocatalytically cleaving ribozyme sequence.

The liposome of the invention, wherein the nucleic acid encodes more than one triple ribozyme is provided. The liposome can comprise any ribozyme-encoding nucleic acid, particularly those described herein.

The viral and liposomal delivery systems of the invention can be used to deliver a nucleic acid comprising a pathogen-specific promoter upstream from a sequence encoding a triple ribozyme comprising a) a 5′ autocatalytically cleaving ribozyme sequence, b) a catalytic ribozyme comprising a target RNA-specific binding site and c) a 3′ autocatalytically cleaving ribozyme sequence.

The nucleic acid delivered by the virion or liposome can encode more than one triple ribozyme. The nucleic acid can encode at least two different triple ribozymes. The nucleic acid can encode more than one copy of the same triple ribozyme. The nucleic acid can encode combinations of different ribozymes, some or all of which may be encoded in more than one copy.

The nucleic acid, wherein at least one triple ribozyme is targeted to the rpoA transcript of the pathogen is provided. The nucleic acid, wherein at least one triple ribozyme is targeted to the secA transcript of the pathogen is provided. The nucleic acid, wherein at least one triple ribozyme is directed to the dnaG transcript of the pathogen is provided. The nucleic acid, wherein at least one triple ribozyme is directed to the ftsz transcript of the pathogen is provided. A ribozyme-encoding nucleic acid can encode all or some of the above triple ribozymes. The triple ribozymes can all be under the control of a single promoter.

Many examples of the nucleic acid encoding the transacting ribozyme of the triple ribozyme are described herein and in the Sequence Listing (e.g., SEQ ID NOS:8-17).

A method of treating an infection in a subject is provided, comprising administering to the subject a virion comprising DNA comprising a target-specific promoter and encoding a ribozyme, whereby the ribozyme encoded by the non-phage DNA is expressed and the infectious agent is killed or weakened. The infection can be bacterial, fungal, yeast, parasitic, viral or non-viral.

Examples of bacterial pathogens that can be targeted by the present virion construct include, but are not limited to, species of the following genera: Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Pasteurella, Clostridium, Corynebacterium, Listeria, Bacillus, Erysipelothrix, Rhodococcus, Escherichia, Klebsiella, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia, Rochlimeae and any other bacterial species now known or later identified to be pathogenic.

The pathogen of the present invention can also include, but is not limited to pathogenic species of yeast/fungal genera (e.g., Candida, Cryptococcus, Aspergillus, Trichophyton, Microsporum)as well as any other yeast or fungus now known or later identified to be pathogenic. Furthermore, the pathogen of the present invention can be a parasite, including, but not limited to, members of the Apicomplexa phylum such as, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp.; Pneumocystis carinii; members of the Microspora phylum such as, for example, Nosema, Enterocytozoon, Encephalitozoon, Septata, Mrazekia, Amblyospora, Ameson, Glugea, Pleistophora and Microsporidium spp.; and members of the Ascetospora phylum such as, for example, Haplosporidium spp., as well as any other parasite now known or later identified to be pathogenic.

Examples of viral pathogens include, but are not limited to, retroviruses (human immunodeficiency viruses), herpesviruses (herpes simplex virus; Epstein Barr virus; varicella zoster virus), orthomyxoviruses (influenza), paramyxoviruses (measles virus; mumps virus; respiratory syncytial virus), picornaviruses (Coxsackie viruses; rhinoviruses), hepatitis viruses (hepatitis C), bunyaviruses (hantavirus; Rift Valley fever virus), arenaviruses (Lassa fever virus), flaviviruses (dengue fever virus; yellow fever virus; chikungunya virus), adenoviruses, birnaviruses, phleboviruses, caliciviruses, hepadnaviruses, orbiviruses, papovaviruses, poxviruses, reoviruses, rotaviruses, rhabdoviruses, parvoviruses, alphaviruses, pestiviruses, rubiviruses, filiviruses, coronaviruses and any other virus now known or later identified to be pathogenic.

The virion construct used in this method can comprise any ribozyme-encoding nucleic acid, particularly those described herein targeted to genes of the pathogen. The virion can be a bacteriophage, or other virus selected for its ability to target a specific cell-type, microorganism or animal. The bacteriophage can be lambda, P1 or other phage. When P1 is the virion, the non-viral DNA can further comprise a PAC site is also provided. This construct is preferred when using P1. Alternatively, the virion can be selected because it has a broad range of targets.

A method of treating an infection in a subject is provided, comprising administering to the subject the liposome comprising DNA comprising a target-specific promoter and encoding a ribozyme, whereby the ribozyme encoded by the DNA is expressed and the infectious agent is killed or weakened. The liposome used in this method can comprise any ribozyme-encoding nucleic acid, particularly those described herein targeted to genes of the pathogen. The infection can be bacterial, fungal, yeast, parasitic, viral or non-viral.

A method of delivering a ribozyme to a target (e.g., a pathogen) in a subject is provided, comprising a) generating a virion comprising DNA having a promoter specific for the target (e.g., the pathogen) and encoding the ribozyme; and b) delivering the virion to the subject, whereby the pathogen-specific promoter directs transcription of the ribozyme in the cells of the pathogen, thereby targeting the ribozyme to the pathogen. The target can be a pathogen, for example, a bacteria, fungus, yeast, parasite, virus or non-viral pathogen.

The above targeting method, wherein the virion is a bacteriophage is provided. The bacteriophage can be lambda, P1 or other phage. The targeting method, wherein the non-viral DNA further comprises a PAC site is also provided. This construct is preferred when using P1.

A method of delivering a ribozyme to a target (e.g., a pathogen) in a subject is provided, comprising a) generating a liposome comprising a promoter and ribozyme encoding sequence; and b) delivering the liposome to the subject, whereby the target-specific promoter directs transcription of the ribozyme in the cells of the target. The target can be a pathogen, for example, a bacteria, fungus, yeast, parasite, virus or non-viral pathogen.

A method of targeted delivery of a ribozyme to a pathogen in a subject, comprising a) generating a virion comprising non-viral DNA of the invention; b) combining it with a liposome; and b) delivering the liposome containing the virion to the subject, whereby liposome enters the eukaryotic cell and releases the virion, which delivers the DNA to the pathogen, whereby the pathogen-specific promoter directs transcription of the ribozyme in the cells of the pathogen.

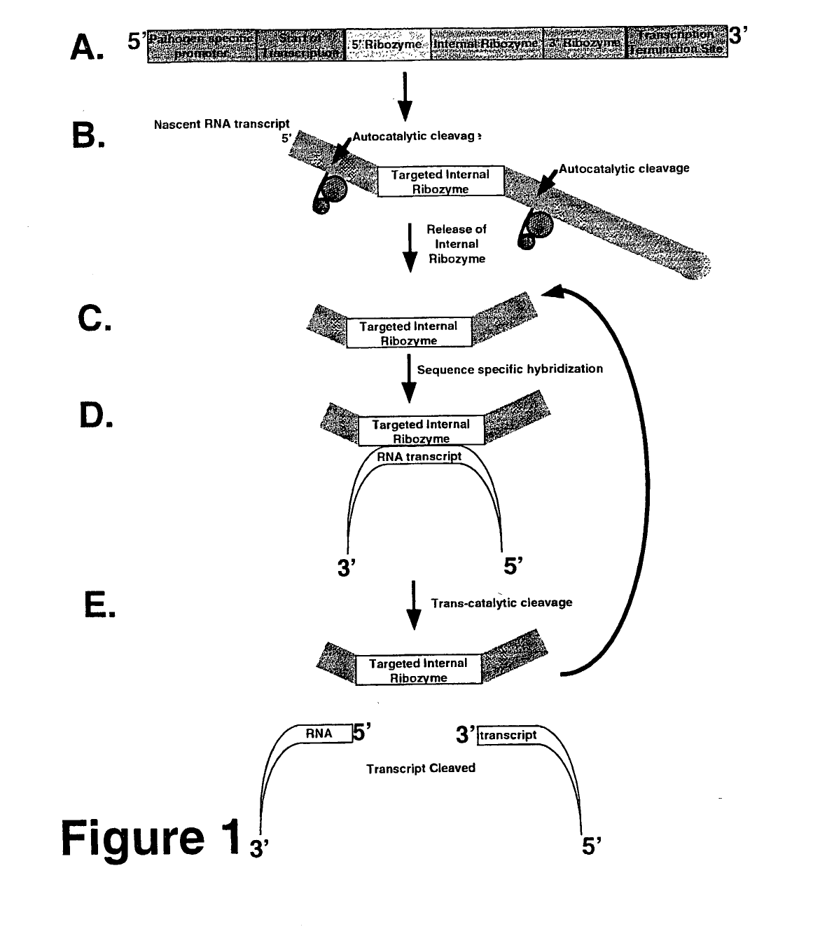

Ribozymes are catalytic RNA molecules that cleave RNA in a sequence specific manner (3, 5, 24). With the invention of the present delivery system and construct, they are uniquely suited as antimicrobial compounds in that they possess an intrinsic enzymatic activity under which the ribozyme can act as a nuclease against itself (cis acting) or against a different RNA targets (trans acting). Catalytic activity of the trans acting ribozyme requires hybridization between the ribozyme and its target at which a catalytic topology is achieved and cleavage occurs. Consequently when three hammerhead ribozymes are assembled in a sequence where the first (5′) and last (3′) ribozyme function as cis acting elements, the endonuclease activity of these 5′ and 3′ ribozymes releases the internal ribozyme whose endonuclease specificity has been targeted against an essential RNA molecule of the invading microorganism (FIG. 1). Thus, the internal ribozyme if this triple ribozyme functions in trans and renders the invading microorganism's RNA(s) inactive through the endonuclease cleavage of the molecule thus destroying the function of the targeted RNA molecule. Expression of the trans acting ribozyme can be achieved by placing the coding sequence of the three hammerhead ribozymes under the control of one or more of the following genetic elements: a naturally occurring strong, intermediate or weak constitutively expressed or regulated promoter from the targeted microorganism, or an artificially contrived constitutively expressed or regulated promoter containing either a strong, intermediate or weak consensus sequence that delivers desired levels of ribozyme expression in the targeted microbe. Once delivered and transcribed the trans acting ribozyme is lethal against the targeted microorganism for it is synthesized at a concentration sufficient to locate and hybridize to all or substantially all of the targeted transcripts. Once hybridization of the internal ribozyme has occurred with the targeted mRNA transcript the internal ribozyme achieves a catalytic topology and cleaves the targeted RNA. Following cleavage the ribozyme is available to repeat the process.

Target Selection

The first critical component in the assembly of the RiboZide™ is the selection of appropriate RNA targets. For ribozymes to be effective anti-microbial therapy, it is preferable to target the RNA of, for example, several key proteins, tRNA, rRNA or any other RNA molecule essential for cell viability or fitness, in order to insure complete inactivation and prevent escape of the invading microorganism. For example, four bacterial genes, essential for viability and unrelated in activity, have been selected and are described herein to highlight how the selection of appropriate mRNA targets is carried out for the preferred construction of the RiboZide™ against prokaryotic targets. Cross-genera RNA targets can be used to design a RiboZide™ that can have broad application, modified by the specificity of the promoter.

In one embodiment of the invention, the first ribozyme targets an essential transcription factor, the second ribozyme targets an essential general secretory component, the third ribozyme targets an essential component of the primosome required for DNA biosynthesis and the fourth ribozyme targets an enzyme required for cell division. Consequently, the ribozymes are redundant in the fact that they inhibit growth by specifically targeting a fundamental process required for bacterial growth. Thus, this can minimize the development of resistance to the antimicrobial therapeutic.

The first gene, rpoA, produces an essential protein, RpoA or the alpha subunit of RNA core polymerase. RpoA was selected rather than the other components of the RNA polymerase holoenzyme, because it is thought to facilitate the assembly of an active RNA Polymerase enzyme complex. Inactivation of the rpoA transcript results in a decrease in the intracellular concentration of the holoenzyme RNA polymerase rendering the cell less able to respond to changes demanded of it once it has invaded a new host. The nucleotide sequence of rpoA is known for a large number of microorganisms (>20 genera) and they are readily available from GenBank.

The second ribozyme target can be the mRNA of the secA gene from bacteria. The product of this gene is the essential and rate-limiting component of the general secretory pathway in bacteria (2). SecA has been found in every prokaryotic cell investigated to date. Additionally, its biosynthesis is translationally coupled to the upstream gene, X (17), presenting a convenient target for a ribozyme. Inhibition or decreased synthesis of seca is also sufficient to confer a reduction in viability to the cell (18). Furthermore, as a pathogen responds to changes required of the infectious process a change in the availability of a key protein such as SecA will disadvantage the pathogen enabling the host to counteract it. Finally, control over the secretion-responsive expression of SecA is at the level of translation (5), and the regulatory sequences within its polycistronic message have been localized to a region comprised of the end of the upstream gene, X, and the beginning of secA. Consequently, inactivation of the transcript by the catalytic cleavage of a ribozyme has profound consequences for the viability of the invading microorganism.

The third ribozyme can target an essential factor for DNA biosynthesis, DnaG. Every 1 to 2 seconds, at least 1,000 times for each replication fork within E. coli, priming of an Okazaki fragment is repeated as a result of an interaction between the cellular primase DnaG (4) and DnaB (11). As would be expected of protein required every 1 to 2 seconds during replication, a lesion within dnaG or an alteration in its concentration results in an immediate stop phenotype (11, 28). Therefore, inactivation of the dnaG message by a ribozyme should have profound cellular consequences in that general priming of the lagging strand is reduced if not eliminated. DnaG is a component of the primosome, a multi-protein complex responsible for priming replication. Any of the components of the primosome, either individually or in any combination, can serve as a target for inactivation of the primosome and, thus, kill the cell. The other components of the primosome are DnaB, DnaC, DnaT, PriA, PriB, and PriC. Thus, the primosome is also sufficiently complex to provide numerous other targets (DnaB, DnaC, DnaT, PriA, PriB and PriC) for inactivation by the trans ribozyme.

The fourth target can be ftsz. This gene also encodes an essential protein, FtsZ, that is required for cell division in that it is responsible for the initiation of separation. FtsZ was selected because its synthesis was under the control of an antisense RNA molecule encoded by the gene dicF. Transcription of dicF is all that is needed to inhibit the translation of ftsZ; thus, overexpression of this antisense molecule is sufficient to cause an inhibition of cell division and a reduction in viability. There is an advantage of using a ribozyme against ftsZ over the antisense molecule, dicF. Specifically, the ribozyme functions catalytically while dicF functions stoichiometrically. Thus, upon cleavage of the ftsZ message the ribozyme attacks additional copies of ftsZ inhibiting the division of the cell. The nucleotide sequence of ftsz like the other targets selected, is commonly available from GenBank.

It should be clear that any other essential protein of a pathogen can have its message targeted in the present invention, and that determining which proteins are essential can be routinely determined according to standard protocols in the art. In fact, there are over 52,000 viral, 41,000 bacterial and 12,300 fungal sequences deposited in the public section of the Entrez Database at the National Center for Biotechnology Information. Any of these can be used to design the catalytic trans ribozyme of the RiboZide. Thus, RiboZid™ can comprise ribozymes targeted to these other messages.

In addition to targeting mRNA of essential proteins ribozymes may be targeted against other RNA species within the cell. Specifically, appropriate targets in bacteria, fungi and other lower eukarytoes include ribosomal RNA such as Small Subunit RNAs (SSU) or Large Subunit (LSU) and tRNA molecules required for protein synthesis. As long as the RNA targeted contains a canonical ribozyme cleavage domain the RiboZide™ therapeutic can hybridize and cleave the RNA complimentary RNA and impact on the fitness of the microbial cell. Additionally, over 3000 rRNA species have been sequenced and aligned. This information is available from the Ribosomal Database Project and should facilitate rapid design and adaptation of ribozyme(s) against such targets. For example the 16S rRNA molecule of bacteria is especially attractive in that there are over 4000 copies of the 16S rRNA per cell. Consequently, a reduction in number slows the process of protein synthesis in so far as the 16S rRNA molecule is involved in the process of translational initiation. Thus, a RiboZide™ containing ribozymes directed against mRNA and rRNA impacts the fitness of the offending microorganism.

Promoter Selection

Promoter selection is accomplished using techniques that are available in the art. For example a method is described in the Examples that permits the selection of both controlled and uncontrolled promoters, as well as consensus promoters that can be design for application in the present RiboZide™. The promoter can be a naturally occurring strong, intermediate or weak constitutively expressed or regulated promoter from the targeted microorganism, or an artificially contrived constitutively expressed or regulated promoter containing either a strong, intermediate or weak consensus sequence that delivers desired levels of ribozyme expression in the targeted microbe.

Ribozyme Design

The RiboZide™ ribozyme possesses sufficient catalytic activity to inactivate the RNA of the targeted transcripts. From an antimicrobial perspective, hammerhead-type ribozymes are especially attractive since the molecule inactivates gene expression catalytically through the cleavage of the phosphodiester bond of the mRNA. Furthermore, hammerhead-type ribozymes have been re-engineered to function in an intermolecular or transducer (trans) acting state (6, 27). The catalytic activity of the ribozyme requires a sufficient concentration of the divalent cation, Mg⁺², and substrate. The substrate can have any sequence as long as the cleavages site contains the recognition element NUX, where N represents any nucleotide, U corresponds to uracil, and X is any nucleotide except G (8). Ribozymes have been widely demonstrated to function in vivo (5,7). The present invention improves the initial design of hammerhead-type ribozymes (25) by constructing a cassette containing two cis acting hammerhead ribozymes flanking a ribozyme that inactivates the targeted RNA. Upon transcription the targeted ribozyme is released as a 60-70 base transcript which not only improves its specificity by reducing non-specific interactions but also improves its catalytic activity as well.

This invention includes modifications to the ribozyme described in U.S. Ser. No. 08/554,369. The arms of the cis acting ribozymes have been lengthened by 20 bases. The sequence has been modified to enhance the catalytic activity of the cis-acting elements, for example those shown in SEQ ID NOS:18-38. Additional restriction sites are included that facilitate easier cloning and manufacturing. tRNA elements are present in to the 3′ end of the RiboZide™ . The addition of the tRNA elements creates additional structure that improves the stability of RiboZide™ helping it resist nuclease attack. An inverted nucleotide repeat has been inserted into the 3′ end of the RiboZide™. The addition of the inverted repeat, a hairpin loop structure, improves the stability of RiboZide™ helping it resist nuclease attack.

Assembly of the Ribozyme(s) into the RiboZide™

Until the present invention, the therapeutic use of ribozymes in eukaryotes was limited because a convenient and efficient delivery system has not been available. A key to the present invention is the strategies used to deliver the ribozyme to the targeted microorganism. Two separate classes of delivery systems can be manufactured, one biologic in nature and the other abiologic.

The key features of the present invention are the combination of ribozyme with viral delivery and assembly of the virions using a unique combination of plasmid features.

Abiologic Delivery Vehicles

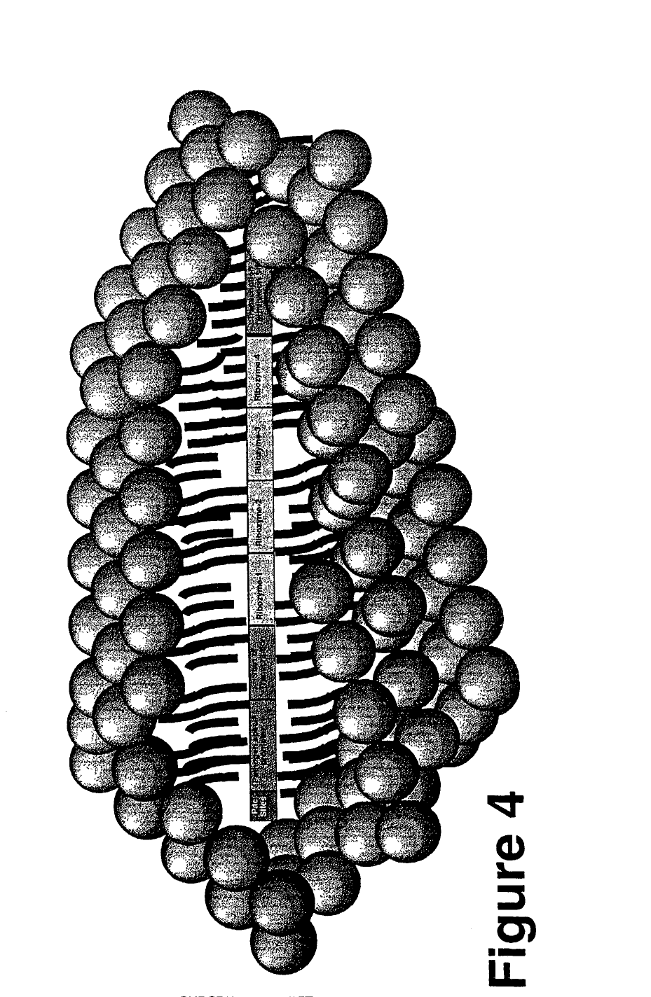

Abiologic delivery of the ribozyme is accomplished by packaging plasmid DNA carrying the gene(s) that codes for the ribozyme(s) into liposomes (FIG. 4.). The liposome is be composed of cationic and neutral lipids commonly used to transfect cells in vitro. The cationic lipids complex with the plasmid DNA and form liposomes.

The RiboZide that is administered to a subject can further comprise a liposome. Cationic and neutral liposomes are contemplated by this invention. Cationic liposomes can be complexed with the a negatively-charged biologically active molecule (e.g., DNA) by mixing these components and allowing them to charge-associate. Cationic liposomes are particularly useful when the biologically active molecule is a nucleic acid because of the nucleic acids negative charge. Examples of cationic liposomes include lipofectin, lipofectamine, lipofectace and DOTAP (30-32). Procedures for forming cationic liposomes encasing substances are standard in the art (33) and can readily be utilized herein by one of ordinary skill in the art to encase the complex of this invention.

The liposomes may be incorporated into a topical ointment for application or delivered in other forms, such as a solution which can be injected into an abscess or delivered systemically.

Plasmid DNA coding for the ribozymes is used rather than preformed ribozymes for the following reasons. Plasmid DNA allows the targeted cells to produce the ribozyme and, thus, results in a higher delivered dose to the cell than can be expected by delivery of ribozyme RNA via liposome. The DNA also provides specificity of action based on target sequence specificity. The liposomes deliver their DNA to any cell in the area of administration, including cells of the host. The promoter driving the transcription of the ribozyme is specific for the targeted microorganism and, thus, will be inactive in other cell types. Therefore, liposomal delivery of DNA coding for the ribozyme provides amplification and specificity.

Biologic Delivery Vehicles

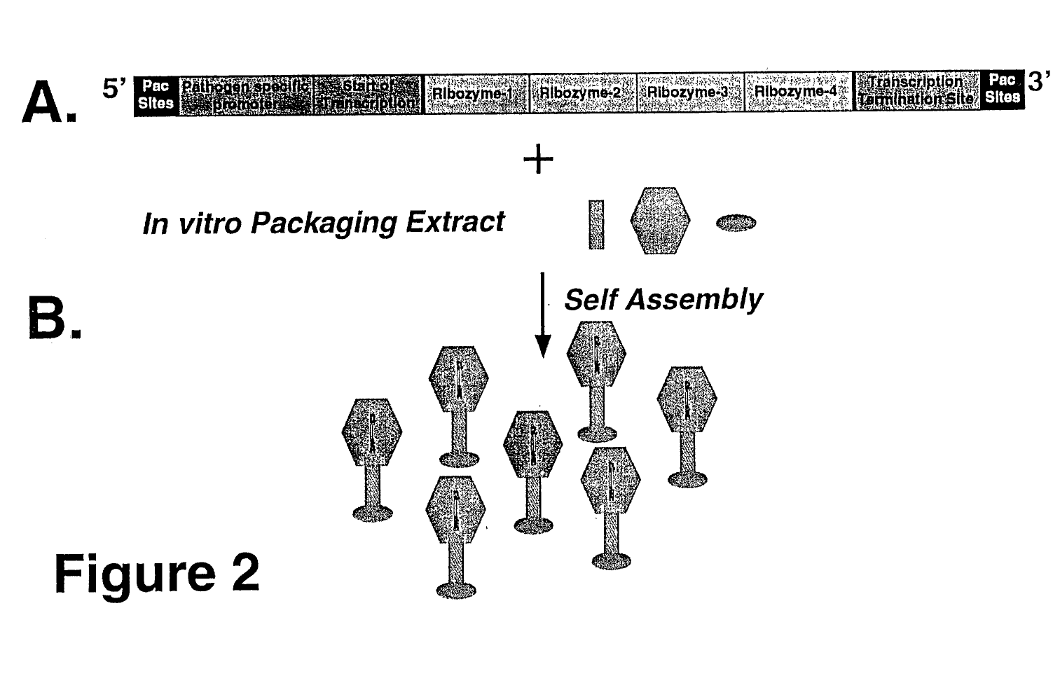

Not all microorganisms are expected to take up DNA delivered by liposome. Consequently, a biologic delivery system is also required. The biologic delivery vehicle of the RiboZide™ takes advantage of the fact that generalized transducing particles completely lack DNA originating from the viral vector. Instead such particles only contain sequences of host origin. Consequently, the invention uses a biologic assembly of viral head proteins (packaging elements for the antimicrobial therapeutic) around the nucleic acid containing the necessary genetic elements that will insure the desired level of expression of the ribozyme(s) (FIGS. 2, 3 and 4).

This delivery system consists of a DNA plasmid carrying the gene(s) coding for the ribozyme(s) packaged into viral particles. Specificity is conferred by the promoter driving transcription of the ribozymes and by the host specificity of the viral vehicle. The following description is one example of the system using bacteriophage lambda virions to package DNA carrying ribozymes directed against Escherichia coli. Similar strategies are used to generate RiboZides™ capable of delivering ribozymes directed against other microorganisms. The virions used to package the DNA can be species specific, such as the virion derived from the bacteriophage lambda coat, or they can possess a broader host range, such as virion derived from bacteriophage P1. Broad host-range viruses facilitate production of the anti-microbial agents without the loss of species specificity because species-specific promoters are used to direct the transcription of the ribozymes which are directed against species specific targeted RNA sequences.

One example of construction the present RibZide entails the use of a plasmid carrying the ribozyme gene(s), a plasmid origin of replication, a selectable marker for plasmid maintenance, the minimal lambda origin of replication, and cos sites, which are required for packaging of DNA into lambda virions. This plasmid is maintained in a lambda lysogen that is defective in integration/excision and recombination functions. The defective lysogen provides all of the replication factors needed to activate the lambda origin of replication on the plasmid and all of the structural components needed to form mature virions; however, the lysogen is not able to replicate and package its own DNA into the virions. The lysogen also carries the cI⁸⁵⁷ temperature-sensitive repressor mutation. Induction of the lysogen is described in the Examples. A similar strategy can be used to generate ribozyme-encoding plasmids packaged into bacteriophage PI virions.

A common bacteriophage of E. coli, is an attractive delivery vehicle for the invention, RiboZide for a number of reasons. First and foremost, P1 has a broad intergenera and interspecies range (29). The P1 receptor of E. coli is the terminal glucose of the lipopolysaccharide (LPS) core lysergic ring of the bacterial outer membrane (12). Yarmolinsky and Sternberg report that in addition to E. coli, this particular phage has the ability to inject its nucleic acid into a large number (>25) of diverse gram negative bacteria (29). Secondly, P1 can accommodate a significant amount of genetic information, over 2% (100,000 bp) of the DNA of E. coli (12). Consequently, gene dosage of the ribozymes can be increased through multiplication of the present ribozyme cassettes, thereby increasing the microbiocidal activity of the RiboZide™. Bacterial strains already exist that can be readily modified to package ribozyme coding DNA in vivo by a process similar to that described above. Additionally, a process utilizing in vitro packaging is also possible. In vitro packaging can be accomplished through the addition of PAC-sites to the genetic information already present within the ribozyme construct. P1 packaging initiates within one of the P1 PAC genes (23). It has been reported that the active PAC site is contained within a 161 base-pair segment of the P1 EcoR1 fragment 20 (23). Thus, the phage head serves as a molecular syringe that delivers the inactivating ribozyme(s) to the pathogen.

Protection of RiboZide-Producing Cells

The genes coding for the ribozymes can be toxic to the cells that are needed to produce the ribozyme-carrying virions. When using a broad host-range virus like P1, the organism used to produce the RiboZide™ can be different from the target organism. In this way, the producing strain is resistant to the toxic effects of the ribozymes because the ribozymes are not efficiently expressed in the producing strain, due to species-specific promoter elements, and the ribozymes will not have any target RNA molecules to attack, due to the species-specific sequences that target the ribozymes. When using a species-specific virus that must be expressed and assembled within a strain of the targeted microorganism, this toxicity becomes a significant concern. The assembly of a RiboZide™ consisting of anti-E. coli ribozyme genes packaged in lambda will illustrate the approach used to circumvent the toxicity. The ribozymes directed against RNA species of E. coli is expressed from a artificial promoter containing consensus promoter elements. This promoter provides high level transcription of the ribozyme immediately upon infection of targeted cells. In order to prevent the unwanted death of the producing strain of E. coli, transcription is repressed in the producing strain by a mechanism not available to the wildtype strains that are targeted for killing. Sequences constituting the DNA binding sites for a heterologous transcription factor are interspersed between the essential activating elements of the ribozyme promoter. Expression of the heterologous transcription factor in the producing strain results in the occlusion of the activating promoter elements and preventing the binding of RNA polymerase. As an example, the gene for the Saccharomyces cerevisiae transcription factor Ste12p may be expressed in E. coli and bind to its binding sites, the pheromone response element, located within the ribozyme promoter. Ste12p will not be found in wild strains of E. coli; therefore, the ribozyme promoter will be accessible to RNA polymerase following delivery of the plasmid to the targeted cells.

An alternative strategy that can protect the producing strain from the toxicity of the ribozymes employs ribozyme-resistant versions of the targeted RNA molecules. This strategy can be used when the target RNA molecule codes for a protein. The ribozyme target site within the mRNA molecule is mutated by site-directed mutagenesis such that the amino acid sequence of the translated protein does not change but the mRNA sequence no longer serves as a substrate for the ribozyme. For example, hammerhead ribozymes require an NUX sequence within the target mRNA for cleavage to occur. By changing this sequence to something else, the ribozyme will not cleave the mRNA. This type of ribozyme resistant version of the target RNA can be expressed from a plasmid or integrated into the chromosome of the producing strain and thus render this strain resistant to the toxic effects of the ribozyme.

Merrill and co-workers recently reported on the selection of long-circulating bacteriophages as anti-bacterial agents (13). They were able to show that it is possible to select for phage variants that remain refractory to clearance by the reticuloendothelial system (RESONANT) for a period of time sufficient to confer a therapeutic response within an infected animal (13). Specifically, the phage will promptly induce neutralizing antibodies interfering with the phage's ability to attack against the bacteria and opsonins that will restore the vulnerability of the phage to the RESONANT (10). The improvement in the present invention is that a non-replicative delivery system has an advantage in that once the phage coat has injected the nucleic acid into the targeted bacterium, the expression of the RiboZide™ ribozyme will destroy the microbe, as opposed to a lytic infection cycle typical of an intact bacteriophage. Consequently, amplification of the phage coat will not be an issue and it is less likely that the non-replicative phage delivery system will generate an immune response such that subsequent use of the delivery system would be jeopardized. Moreover, if the patient has been exposed to a resistant pathogenic microbe and the RiboZide™ is effective and neutralizes the invading microbe, then it is expected that the microbial antigens liberated as a result of the action of the RiboZide™, will illicit sufficient humoral immunity and cell-mediated immunity to confer protection against subsequent attacks.

Administration

Parenteral administration, if used, is generally characterized by injection (intravenous, intradermal, subcutaneous and intramuscular). Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant level of dosage is maintained. See, e.g., U.S. Pat. No. 3,610,795, which is incorporated by reference herein.

Suitable carriers for parenteral administration of the substance in a sterile solution or suspension can include sterile saline that can contain additives, such as ethyl oleate or isopropyl myristate, and can be injected, for example, intravenously, as well as into subcutaneous or intramuscular tissues.

Topical administration can be by creams, gels, suppositories and the like. Ex vivo (extracorporeal) delivery can be as typically used in other contexts. Oral administration is also contemplated.

Suitable carriers for oral administration include one or more substances which can also act as flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers can be water, pyrogen free saline, pharmaceutically accepted oils, or a mixture of any of these. The liquid can also contain other suitable pharmaceutical additions such as buffers, preservatives, flavoring agents, viscosity or osmo-regulators, stabilizers or suspending agents. Examples of suitable liquid carriers include water with or without various additives, including carboxypolymethylene as a pH-regulated gel.

The RiboZide™ can be administered to the subject in amounts sufficient to produce an antibiotic effect or to inhibit or reduce the activity of the target pathogen. Optimal dosages used will vary according to the individual, on the basis of age, size, weight, condition, etc, as well as the particular modulating effect being induced. One skilled in the art will realize that dosages are best optimized by the practicing physician and methods for determining dosage are described, for example, in Remington's Pharmaceutical Sciences[Martin, E. W. (ed.) Remington's Pharmaceutical Sciences, latest edition Mack Publishing Co., Easton, Pa.]. Treatment can be at intervals and can be continued for an indefinite period of time, as indicated by monitoring of the signs, symptoms and clinical parameters associated with a particular infection. The parameters associated with infection are well known for many pathogens and can be routinely assessed during the course treatment.

### EXAMPLES

Promoter Selection

Promoters specific for the target (e.g., a specific pathogen, genus, etc.) in question can be selected by screening genomic sequences for the ability to activate a promoterless reporter gene. The promoterless reporter gene is based on the strategy developed for use with plasmid pMC1871 (Casadaban M J et al Meth. Enzymol. 100, 293 (1983)). For non-viral pathogens, plasmid capable of stable replication and maintenance in the microorganism understudy is modified by standard molecular biology techniques to carry the coding region of a reporter gene (Sambrook et al. latest eddition). The reporter gene can be any of a number of standard reporter genes including but not limited to the lacZ gene of E. coli, which codes for β-galactosidase. Total genomic DNA is isolated from cells of the pathogen, cleaved with restriction endonucleases to yield fragments of a few hundred basepairs on average. These fragments are then ligated into a unique restriction endonuclease cleavage site at the 5′end of the reporter gene coding region, creating a library of plasmids. The library is then transformed into the pathogen by standard techniques and the resulting transformants are screened for expression of the reporter gene. In the case of lacZ, the transformants can be plated onto medium containing the chromogenic ^{o}-galactosidase substrate X-Gal (5-bromo-4-chloro-3-indolyl-^{o}-D-galactoside). Transformants that contain a plasmid with an insert carrying a promoter will express β-galactosidase and will turn blue on X-Gal plates. The intensity of the blue color is relative to the level of expression; promoters of different strength can be selected based on the intensity of the blue color.

The above-described screening procedure can be modified to identify regulated promoters. For example, promoters that are regulated by carbon source availability can be screened on plates that contain different carbon sources. Other modifications are possible and will depend, in part, on the organism in question. To test for species-specificity, the identified promoters are transferred to promoterless reporter plasmids capable of replication and maintenance in a different organism. Truly species-specific promoters will not activate the expression of the reporter gene in any other species. Obvious modifications can be used to identify and test artificial promoters composed of synthetic oligonucleotides inserted into the promoterless reporter plasmid.

Biologic Delivery.

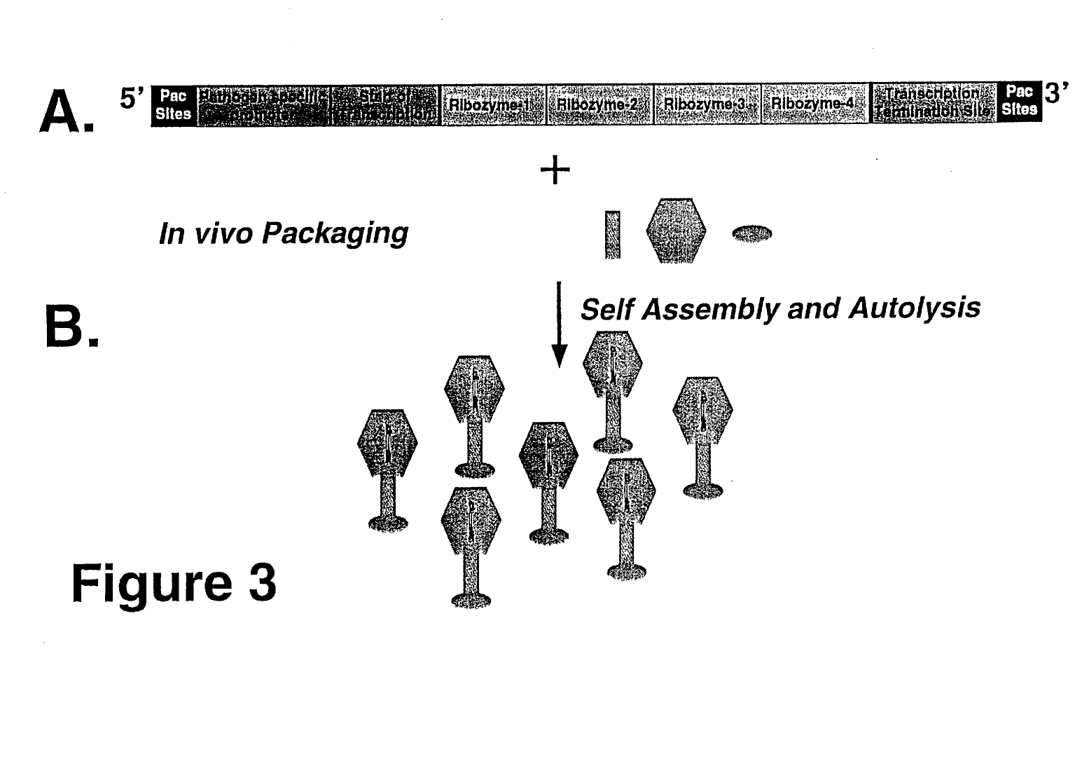

The present example entails the use of a plasmid carrying the ribozyme gene(s), a plasmid origin of replication, a selectable marker for plasmid maintenance, the minimal lambda origin of replication, and cos sites, which are required for packaging of DNA into lambda virions. This plasmid is maintained in a lambda lysogen that is defective in integration/excision and recombination functions. The defective lysogen provides all of the replication factors needed to activate the lambda origin of replication on the plasmid and all of the structural components needed to form mature virions; however, the lysogen is not able to replicate and package its own DNA into the virions. The lysogen also carries the cI⁸⁵⁷ temperature-sensitive repressor mutation. Induction of the lysogen by temperature shift to 42° C. or by other means, such as exposure to 5J/m² of ultraviolet radiation will mobilize the plasmid and result in its replication and packaging into lambda virions (FIG. 3). The virions can then be harvested, purified free of E. coli proteins and be used to deliver the ribozyme gene(s) to E. coli.

Abiologic Delivery.

Abiologic delivery of the RiboZide™ is accomplished with ribozyme(s) constructs that have been engineered to be expressed within the targeted tissue. Briefly, the genetic element containing the promoter and ribozyme(s) are complexed with cationic liposomes (Lipofectamine—Gibco BRL) in a 1:10 ratio and are introduced into test animals by either single or multiple injection of 0.2 ml total volume nucleic acid-liposome mixture.

In Vivo Testing

Following the demonstration that RiboZides™ have an in vitro biological activity (either directly on bacterial cultures or in an infectious tissue culture cell assay system), the effectiveness of the RiboZides™ is shown in an in vivo model system. To demonstrate the efficacy of RiboZides™ in vivo, experimental animal model systems are utilized. For an initial demonstration of the efficacy of the RiboZides™ in vivo, mice are infected with a microbial pathogen which has previously been shown to be sensitive to the RiboZide™ construct(s) and the effect of RiboZides administered in vivo is determined. In the first series of in vivo trials, one determines the effectiveness of RiboZides™ at preventing an acute infection in a murine model system when the RiboZide™ is added directly to the microbe prior to administration in vivo.

The next series of trials determine whether the administration of RiboZides™ after infection is effective at preventing an acute bacterial infections. In addition to the clinical status of infected mice, tissues obtained at necropsy are examined histologically and the presence of replicating microorganism in tissue samples is determined by standard methodology. Animals can be infected by various routes (systemic and/or mucosal) and the RiboZides™ delivered over time after infection by systemic and/or mucosal routes. Both abiologic as well as biological delivery of RiboZides™ is used. The demonstration of a positive effect of the RiboZides™ in controlled experimental model system provides compelling evidence for the efficacy of the preparation and determines whether or not the preparation warrants evaluation under conditions of standard clinical trials.

Development and Testing of the Catalytic Component of the RiboZideJ

The following is a routine approach for designing, manufacturing and testing of the ribozymes that are incorporated into the RiboZideJ invention.

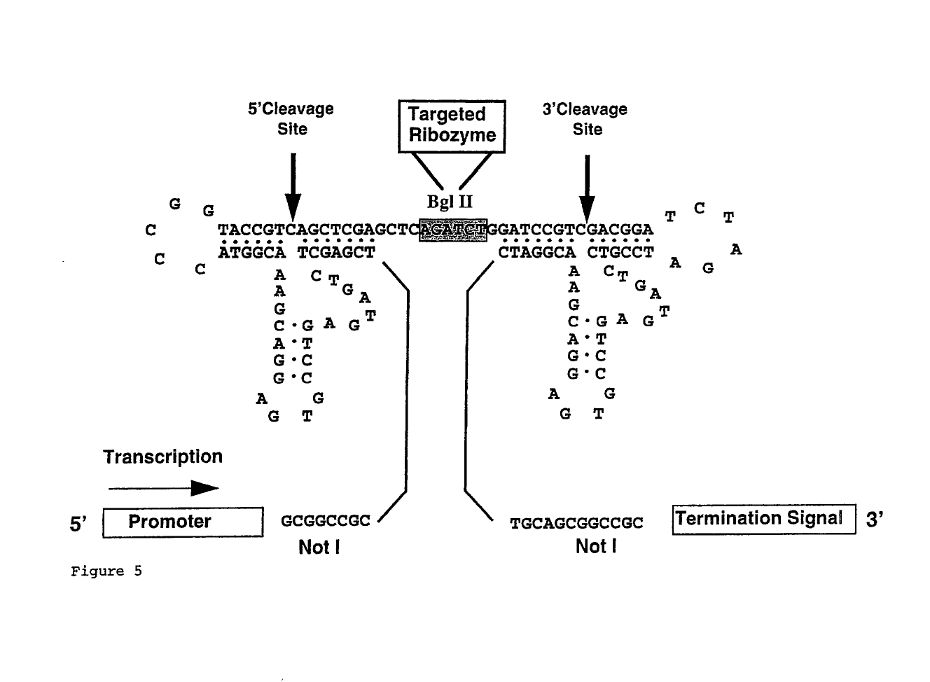

The catalytic component of RiboZidej invention is/are transacting internal targeted ribozyme(s) (ITRz). To facilitate construction of this critical and catalytic component, the targeted triple ribozyme (TRz) containing a double ribozyme cassette was developed: This artifically contrived genetic element consists of autocatalytic, self-cleaving 5′ and 3′ ribozymes (FIG. 5), with a cloning region (denoted by the box entitled Targeted Ribozyme) between them. This double ribozyme cassette is then placed within a series of expression vectors that were either constructed (pClip), purchased from commercial vendors (pBluescriptII, Stratagene; pCRII, InVitrogen; pET-30a-c; pBACsurf-1, pIE1 and pIE4, Novagen) and used intact or modified as necessary to confer the desired activity within the RiboZideJ. pClip (the genetic element described in FIG. 5) is a modification of pBluescript, wherein the cassett shown is dropped into the Not I site in pBluescript. The targeted ribozyme (transacting catalytic ribozyme) is dropped into the Bgl II site (TGCTCT).

An internal targeted ribozyme (ITRZ) is synthesized as reverse complementary overlapping oligodeoxynucleotides, which are designed in such a way that when annealed they form single stranded ends identical to those produced by digestion with the restriction endonuclease contained with the region between the two cis-acting ribozymes. In this particular example the restriction endonuclease recognition site is that recognized by Bgl II. Essentially any RNA can be targeted: specificity is conferred by selecting sequences for the ribozyme that are reverse and complementary to sequences flanking the chosen cleavage site in the targeted RNA molecule. These internal targeted ribozymes are then cloned into the cloning region within the double ribozyme cassette (FIG. 1) to produce the targeted ribozyme. Internal targeted ribozymes to prokaryotic sequences have been constructed including, but not limited to, Escherichia coli: secA (EcoSecA, AE000119 U00096), gene X (EcoSecA, AE000119 U00096) ftsZ (AE000119;U00096), dnaG (AE000388 U00096) , rpoA(AE000407 U00096) and tRNA-asp (X14007), Streptomyces lividins secA (Z50195), Enterococcus faecalis, ftsZ (U94707), Pseudomonas putida, dnaG (U85774), Streptomyces coelicolor rpoA (X92107), Staphylococcus warneri tRNA-Asp (X66089 S42075).

SEQ ID NOS:1-7 are examples of the transacting ribosyme sequences.

The following sequences are for ribozymes directed against the targets described. The naming system refers to the target cytosine in the GUC motif. It is the nucleotides number from the referenced sequence (accession number indicated). Ribozymes directed against secA targets have restriction sites for Bgl II on both ends. All other inserts have Bgl II (5 end) and Sty I (3 end) restriction sites for use in the new vector. Antisense arms are boldfaced.

Escherichia coli
ftsZ target (ACCESSION: AE000119 U00096)

| | | |
|---|---|---|
| 105 | AGATCTAAACGCCGATCTGAGGAGTCCGTGAGGACGAAACTTTAAAAACCAAGG | (SEQ ID NO:8) |
| 713 | AGATCTAAACATCTCACTGATGAGTCCGTGAGGACGAAACATTACGAAACCAAGG | |
| 1131 | AGATCTAAATCATTCACCTGATGAGTCCGTGAGGACGAAACTTTAGCAAACCAAGG | |

secA target (ACCESSION: AB000119 U00096)

| | | |
|---|---|---|
| 84 | AGATCTAAAAAAAAACCTGATGAGTCCGTGAGGACGAAACTGGTTAAAAGATCT | (SEQ ID NO:9) |
| 707 | AGATCTAAATTATCCACTGATGAGTCCGTGAGGACGAAACGGGCGAAAAGATCT | |
| 856 | AGATCTAAATCGTTACCTGATGAGTCCGTGAGGACGAAACTACCQAAAAGATCT | |
| 894 | AGATCTAAATGATGTTCTGATGAGTCCGTGAGGACGAAACCACTTAAAAAGATCT | |
| 979 | AGATCTAAATTTTCCACTGATGAGTCCGTGAGGACGAAACGTGCAAAAATCT | |
| 1282 | AGATCTAAATTGATACCCTGATGAGTCCGTGAGGACGAAACAGTCAGAAAAGATCT | |
| 2216 | AGATCTAAATTCGTTTCTGATGAGTCCGTGAGGACGAAACACCACAAAAGATCT | |

dnaG target (ACCESSION: AE000388 U00096)

| | | |
|---|---|---|
| 5344 | AGATCTAAACGTTTAGTCTGATGAGTCCGTGAGGACGAAACCAACAAAACCAAGG | (SEQ ID NO:10) |
| 5903 | AGATCTAAAGGCATCACTGATGAGTCCGTGAGGACGAAACTGTTAAAACCAAGG | |
| 6336 | AGATCTAAACCACATCCTGATGAGTCCGTGAGGACGAAACAGTTTAAACCAAGG | |

rpoA target (ACCESSION: AE000407 U00096)

| | | |
|---|---|---|
| 8308 | AGATCTAAAAGAGCGCTGATGAGTCCGTGAGGACGAAACAGTCAAAACCAAGG | (SEQ ID NO:11) |
| 8494 | AGATCTAAATTTCGATCTGATGAGTCCGTGAGGACGAAACCAGCTAAACCAAGG | |
| 8737 | AGATCTAAACGATTTCCTGATGAGTCCGTGAGGACGAAACATCACCAAACCAAGG | |

tRNA-Asp target (directed against GUC anticodon loop. Accession: X14007)

| | | |
|---|---|---|
| 172 | AGATCTAAATGCGTCTGATGAGTCCGTGAGGACGAAACAGGCAGGTAAAACCAAGG | (SEQ ID NO:12) |

Streptomyces lividans
secA target (ACCESSION: Z50195)

| | | |
|---|---|---|
| 1080 | AGATCTAAACTCGTCCTGATGAGTCCGTGAGGACGAAACGATCAAAACCAAGG | (SEQ ID NO:13) |
| 2033 | AGATCTAAAGGGCGCTGATGAGTCCGTGAGGACGAAACGCGAAACCAAGG | |
| 2556 | AGATCTAAAGTACTCCTGATGAGTCCGTGAGGACGAAACCAGCGAAACCAAGG | |

Enterococcus faecalis
ftsZ target (ACCESSION: U94707)

| | | |
|---|---|---|
| 10805 | AGATCTAAAACTAAATGCTGATGAGTCCGTGAGGACGAAACGAGTTAAAACCAAGG | (SEQ ID NO:14) |
| 11182 | AGATCTAAAGTTTAATAACTGATGAGTCCGTGAGGACGAAACTTGTTCAAACCAAGG | |
| 11512 | AGATCTAAAACTTTTGCTGATGAGTCCGTGAGGACGAAACGTGTATAAACCAAGG | |

| | |
|---|---|
| Pseudomonas putida | |
| dnaG target (ACCESSION: | U85774) |

| | | |
|---|---|---|
| 222 | AGATCTAAAGGTCCATCTGATGAGTCCGTGAGGACGAAAGCAAACCAAGG | (SEQ ID NO:15) |
| 986 | AGATCTAAACAGGTTCCTGATGAGTCCGTGAGGACGAAACAATGTAAACCAAGG | |
| 1891 | AGATCTAAATCGCTTTCTGATGAGTCCGTGAGGACGAAACGTGATAAACCAAGG | |

Streptomyces coelicolor
rpoA target (ACCESSION: X92107)

| | | |
|---|---|---|
| 290 | AGATCTAAAGCTCGATCTGATGAGTCCGTGAGGACGAAACGAACCAAACCAAGG | (SEQ ID NO:16) |
| 716 | AGATCTAAACGAGTCCTGATGAGTCCGTGAGGACGAAACCGGGAAACCAAGG | |
| 1099 | AGATCTAAAGTCGATGCTGATGAGTCCGTGAGGACGAAACTTCGCAAACCAAGG | |

Staphylococcus warneri
tRNA-Asp target (directed against GUC anticodon loop. Accession: X66089 S42075)

| | | |
|---|---|---|
| 62 | AGATCTAAATGCGTCTGATGAGTCCGTGAGGACGAAACAGGCAGGCGAAACCAAGG | (SEQ ID NO:17) |

The utility of the design using eukaryotic sequences has also been evaluated; a) repetitive B2 transcripts (B2); b) RNA polymerase I (polI); c) Hepatitis B virus (HBV); d) Sonic Hedgehog (SH); e) Human Papillomavirus E6/E7 protein (HPV); f) RNA polymerase II (polII); g) Insulin-like Growth Factor 1 (IGF1); h) retinoblastoma protein (RB); i) and j) Multicatalytic Proteinase alpha-subunits C3 and C9 (C3 and C9, respectively); k) telomerase (tel); 1) Transforming growth factor beta (TGFβ); m) catalase (CAT); n) Peroxisome proliferation associated receptor (PpaRα); and o) Cytochrome P₄₅₀ 1E1 (p4501E1). Target RNAs (with locus names and accession numbers) as well as the selected target sites are presented (Table 1), as are the sequences of these ITRz (SEQ ID NOS:18-36).

TABLE 1
Summary of Targeted RNAs and Target Sites.

| | | | Target | Functional Testing | |
|---|---|---|---|---|---|
| Target RNA | EMBL Locus | Accession | Site | in vitro | in vivo |
| pol II | HSRNAP14K | Z27113 | GTC₈₃ | ND | ND |
| HBV | XXHEPAV | X02496 | GTC₄₃₈ | IP | + |
| RB | MUSPP105RB | M26391 | GTC₂₆₄ | + | + |
| IGF1 | HUMIGF1B | M37484 | GTC₁₈₅ | ND | ND |
| SH | MMEVX1 | X54239 | GTC₅₅₈ | IP | IP |
| Pol I | MUSRPA40 | D31966 | GTC₄₅₈ | + | + |
| HPV | PPH16 | K02718 | GTT₁₀₈ | IP | + |
| C3 | RATC3AA | J02897 | GTT₂₂ | + | + |
| C9 | RNPTSC9 | X533304 | GTC₁₀₁ | + | + |
| B2 | B2-Consensus | ## | GTT₂₄ | + | + |
| Tel | MMU33831 | U33831 | CTA₆₃ | ND | ND |

| | | |
|---|---|---|
| B2 | TGCTCTT CTGATGAGTCCGTGAGGACGAAA CCGCCTGA | (SEQ ID NO:18) |
| Pol 1 | TTCAAAG CTGATGAGTCCGTGAGGACGAAA CGAGGATC | (SEQ ID NO:19) |
| Sonic | GTCCAT CTGATGAGTCCGTGAGGACGAAA CCGGC | (SEQ ID NO:20) |
| Hedgehog | | |
| HBV | ATTAGAG CTGATGAGTCCGTGAGGACGAAA CAAACG | (SEQ ID NO:21) |
| HPV | GTCCTGA CTGATGAGTCCGTGAGGACGAAA CATTGCA | (SEQ ID NO:22) |
| Pol III | TCCGTTGTCT CTGATGAGTCCGTGAGGACGAAA CATGACACCGA | (SEQ ID NO:23) |
| IGF-1 | GCGAGGAG CTGATGAGTCCGTGAGGACGAAA CATGGTGT | (SEQ ID NO:24) |
| RB | AACTTTT CTGATGAGTCCGTGAGGACGAAA CATAATG | (SEQ ID NO:25) |
| C3 | TCGAAGCTGT CTGATGAGTCCGTGAGGACGAAA CCGCGTTGA | (SEQ ID NO:26) |
| TEL | ATCAGGGT CTGATGAGTCCGTGAGGACGAAA GGTGCC | (Seq ID NO:27) |
| C9 | TCTTCGA CTGATGAGTCCGTGAGGACGAAA CATGGCT | (SEQ ID NO:28) |
| TGFB-1 | TAGCACA CTGATGAGTCCGTGAGGACGAAA CGTTTGA | (SEQ ID NO:29) |
| CAT/#13 | TGCAATA CTGATGAGTCCGTGAGGACGAAA CTGCCT | (SEQ ID NO:30) |
| CAT/#15 | AAGTCAT CTGATGAGTCCGTGAGGACGAAA CCTGGA | (SEQ ID NO:31) |
| PpaRa/#2 | GATAAGG CTGATGAGTCCGTGAGGACGAAA CTTTCC | (SEQ ID NO:32) |
| PpaRa/#B | CATATTC CTGATGAGTCCGTGAGGACGAAA CACTCG | (SEQ ID NO:33) |
| PpaRa/#14 | TCATGTAT CTGATGAGTCCGTGAGGACGAAA CAAAAGG | (SEQ ID NO:34) |
| P4501E1/#2 | GGTTAAA CTGATGAGTCCGTGAGGACGAAA CTTGGG | (SEQ ID NO:35) |
| P4501E1/#B | GTCCAGT CTGATGAGTCCGTGAGGACGAAA CTTAAG | (Seq ID NO:36) |

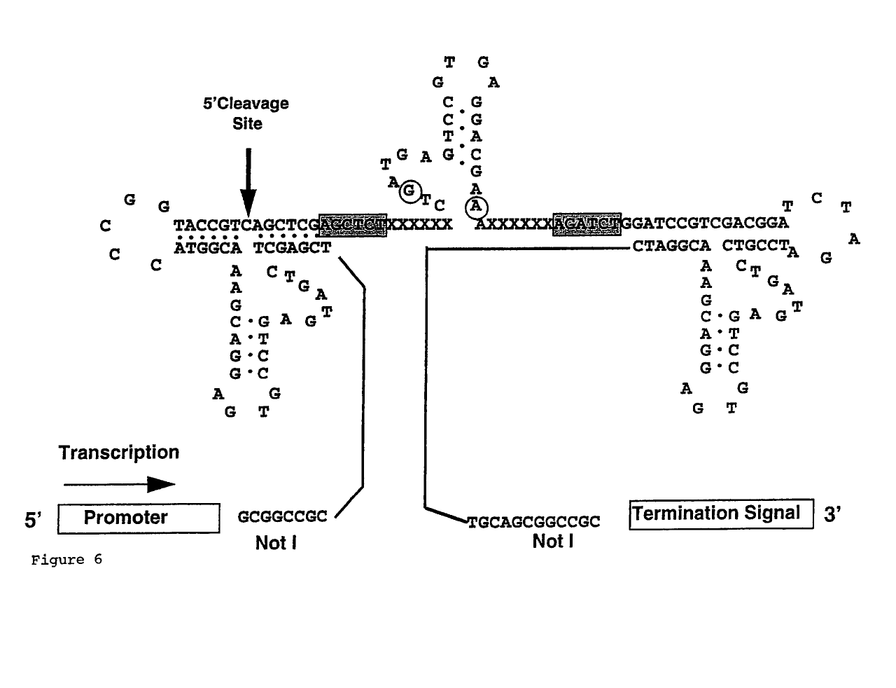

For many of these constructs, “mutants” have also been created by substituting an A for a G, or a G for an A, at nucleotides (denoted within circles, FIG. 6) which are absolutely required for catalytic activity. These “mutants” allow us to document that the efficiency of destruction of the targeted RNAs is due to ribozyme catalytic activity and not to antisense effects.

Characterization and Validation of the TRz Construct

Typically, once the recombinant plasmid has been created the TRz constructs are isolated from the bacterium their nucleotide sequence is determined to confirm their identities and to document their orientation within the vector. The constructs are then transcribed in vitro using SP6 and T7 RNA polymerases with ³²P-CTP. When transcribed in the “sense” orientation, all of these TRz constructs should be “self-liberating”; that is, the 5′ and 3′ self-cleaving ribozymes should work effectively, freeing the ITRz during (or immediately after) transcription (FIG. 1c). The 5′ liberated ribozyme (whose only function is self-cleavage, liberating the 5′ end of the ITRZ) is associated with relatively short stretches of vector sequences and the 3′ self-cleaving ribozyme (whose only function is self-cleavage, liberating the 3′ end of the ITRZ) remains associated with long vector sequences. The liberated ITRz achieves its catalytic topology upon hybridization with the targeted sequence. Transcription of all of these TRz in the “antisense” direction should not result in self-cleavage.

In Vitro Evaluation

Upon validation of the TRz construct, the self-liberating TRz should be evaluated for their ability to effectively cut their targeted RNAs. Appropriate regions of the targeted RNAs are generally cloned using cellular RNA and reverse transcription/polymerase chain reaction amplifications. In some cases, cloned full-length cellular RNAs can also used. The identities of the constructs used for transcription of target RNAs are also confirmed by sequencing. Target RNAs are then synthesized in vitro using the appropriate T7/SP6 RNA polymerase with ³²P-CTP, and are subsequently gel-purified. A preparation of the TRz under evaluation is then synthesized without ³²P-CTP. The TRz preparation is then mixed with their an appropriate concentration of radiometrically ³²P-labeled target or substrate RNA (³²P-labeled target RNAs and unlabeled TRz preparations are mixed at a 10:1 molar ratio) and is incubated for various lengths of time. Following incubations, the RNA is examined using polyacrylamide gel electrophoresis (PAGE) and autoradiography. All of the constructs tested should be able to cleave their target RNAs. In general, the data show an approximate catalytic rate of 0.2 cleavages/ribozyme-min.

In Vivo Evaluation

The TRz should then be evaluated with intact cells. The TRz cassette is excised from the parental plasmid and is then placed into an appropriate expression vector. Vectors utilized include (but are not limited to) the is LacSwitch vector (from Stratagene), which is an IPTG-inducible system, and the TetSplice vector (from Gibco-BRL), which is a tetracycline-inducible system.

The TRz constructs in these expression vectors were then transfected into cells using standard techniques. Cell types used in transfections have included E. coli, human CaSki cervical carcinoma cells, SV-40 immortalized rat hepatocytes, and mouse fibroblasts. In transient transfection analyses, all constructs tested produced substantial reductions in their respective target RNAs.

1. The secA targeted TRz construct against the seca gene of E. coli is in the vector pClip, which is a variation of the generalized cloning vector, pBluescript of Stratagene. The plasmid containing the construct was transformed into competent bacterial cells and cells containing the plasmid with the TRz were selected by using the antibiotic selectable marker within the vector,. pClip. Upon induction of the promoter with isopropyl-β-D thiogalactoside, (IPTG) the effect of ribozyme expression is monitored by standard bacterial viable counts. A reduction in total viable cells is an indication of synthesis and catalytic activity of the TRz against the essential target.

2. The polI-targeted TRz construct (in LacSwitch vector) was used in transfections of SV40-immortalized rat hepatocytes (CWSV1 cells), and stably transfected cell populations were obtained. Cells not transfected with the antibiotic resistance plasmid were all dead by day 5, indicating that the antibiotic selection procedure was effective. Growth of cells transfected with the double ribozyme (as a control, with no cellular RNA target), and of cells expressing the catalytically inactive “mutant” polI TRz, was unaffected. However, growth of cells expressing the polI-targeted TRz was depressed by nearly 90%.. Concurrently, the mRNA for polI was decreased by at least 70% from polI mRNA levels in the cells expressing the mutant polI TRz. Since expression of the TRz was essentially equivalent for the two TRz, this clearly documents that the effects on cell growth are due to TRz catalytic activity and not to antisense effects. In other experiments, expression of the polI-TRz in LacSwitch resulted in cell death with mouse fibroblast cell populations.

3. The RB-targeted TRz (in the tetracycline-inducible TetSplice vector system) was used in transfections with CWSV1 cells, and stably transfected cells were selected using G418 antibiotic in the presence of tetracycline, and individual clones were harvested and used (expression of RB-targeted TRz is “off” in the presence of tetracycline, and is “on” in the absence of tetracycline). Expression of the RB-targeted TRz had no effect on cell growth, as expected. Expression of RB mRNA was substantially reduced to below detectable levels by Northern blot analysis. To extend this result, metabolic labeling of proteins was performed using ³⁵S-methionine, and immunoprecipitations were performed using an antibody directed against RB. The data show that RB protein levels were reduced by 75%, comparing clonal cell populations grown in the presence vs. absence of tetracycline, even after only 18h of induction of RB-targeted TRz expression (removal of tetracycline). This shows that the effects of the reduction in RB RNA levels also extends to the production of the RB protein. In addition, the (+) −tetracycline sample provides an ideal control, since it represents the exact same clonal cell line. Essentially any inducible vector system can be used in parallel fashion.

4. The B2-targeted TRz (in the IPTG-inducible LacSwitch vector) was used in transfections of CWSV1 cells, and antibiotic selection was used to obtain a number of individual clones. Reductions in cytoplasmic B2 RNA levels of up to 80% were observed by Northern blot analysis, and growth of transfected clones was reduced in parallel. In fact, a linear relationship between growth rate and B2 RNA levels was observed. The reductions in B2 transcripts paralleled the level of B2-targeted TRz expression (as determined by slot-blot analysis). The B2 target RNA is of additional interest, because B2 transcripts are not translated (i.e., they are not mRNAs) and they are abundant, highly-structured RNAs.

Other TRz constructs have also been successfully tested using this methodology (including C9 in the tetracycline-inducible system, and CAT, BRACl and Albumin driven by the albumin promoter with HepG2 cells).

Throughout this application various publications are referenced by numbers within parentheses. Full citations for these publications are shown below. The disclosures of these publications in their entireties are hereby incorporated by reference into this application in order to more fully describe the state of the art to which this invention pertains.

### REFERENCES

1. Abdul-Hassan, H. S., A. El-Tahan, B. Massoud, and R. Gomas. 1990. Bacterio phage therapy of pseudomonas burn woud sepis. Annals of the Mediterranean Burn Club. 34:262-264.

2. Bassford, P., J. Beckwith, K. Ito, C. Kumamoto, S. Mizushima, D. Oliver, L. Randall, T. Silhavy, P. C. Tai, and B. Wickner. 1991. The Primary Pathway of Protein Export in E. Coli. Cell. 65(30:367-368.

3. Bertrand E., R. Pictet, and T. Grange. 1994. Can hammerhead ribozymes be efficient tools to inactivate gene function? [published erratum appears in Nucleic Acids Resonant 1994 Apr. 11; 22(7):1326]. Nucleic Acids Resonant. 22(3):293-300.

4. Bouche, J. P., K. Zechel, and A. Kornberg. 1975 dnaG gene product, a rifampicin resistant RNA polymerase, initiates the conversion of a single stranded coliphage DNA to its duplex replicative form. J. Biol. Chem. 250:5995-6001.

5. Christoffersen, R. E., and J. J. Marr. 1995. Ribozymes as human therapeutic agents. J. Med. Chem. 38(12):2023-37.

6. Haseloff, J., and W. L. Gerlach. 1988. Simple RNA enzymes with new and highly specific endoribonuclease activities. Nature. 334(6183):585-91.

7. Inokuchi, Y., N. Yayama, A. Hirashima, S. Nishikawa, J. Ohkawa and K. Taira. 1994. A hammerhead ribozyme inhibits the proliferation of an RNA coliphage SP in Eschirichia coli. J. biol. Chem. 269(15):11361-6.

8. Koizumi, M., Y. Hayase, S. Iwai, H. Kamiya, H. Inoue, and E. Ohtsuka. 1989. Design of RNA enzymes distinguishing a single base mutation in RNA. Nucleic Acids Resonant. 17(17):7059-71.

9. Kwarcinski, W., B. Lzarakiewicz, B. Weber-Dabrwoska, J. Rudnicki, K. Kaminski, and M. Sciebura. 1994. Bakteriofagoterapia w. leczeniu nawracajacego ropnia podprzeponowego i podwatrobowego oraz przetoki jewlitowej Patent-Office wycieciu zoladka. Polski Tygodnik Lekarski. XLIX:23-23.

10. Lederberg, J. 1966. Smaller fleas . . . ad infinitum: Therapeutic bacteriophage redux. Proc. Natl. Acad. Sci. USA 93:3167-3168.

11. Marians, K. J. 1996. Replication Fork Propagation, p. 749-763. In F. C. Neidhardt (ed.), Escherichia coli and Salmonella: Cellular and Molecular Biology, 2nd ed, vol. 1. American Society for Microbiology, Washington, DC.

12. Masters, M. 1966. Generalized Transduction, p. 2421-2441. In F. Neidhardt (ed.), Escherichia coli and Salmonella: Cellular and Molecular Biology, 2d ed. Vol.2, ASM Press, Washington, DC.

13. Merril., C.R., B. Biswas, R. Carlton, N. C. Jensen, and C. J. Creed. 1966. Long-circulating bacteriophage as antibacterial agents. Proc. Natl. Acad. claimed invention. USA. 93:3188-3192.

14. Mizuno, T., M. Y. Chou, and M. Inouye. 1984. A unique mechanism regulating gene expression: translations inhibition by a complementary RNA transcript (micRNA). Proc. Natl. Acad Sci USA. 81(7):1966070.

15. Pace, N. R., and D. Smith. 1990. Riboneuclease P: function and variation. J. Biol. Chem. 256(7):3587-90.

16. Schmidt, M., and N. Delihas. 1995. micF RNA is a substrate for Rnase E.FEMS Microbiol. Lett. 133(3):209-13.

17. Schmidt, M. G., K. M. Dolan, and D. B. Oliver. 199,1. Regulation of Escherichia coli secA mRNA translation by a secretion-responsive element. J. Bacteriol. 173(20):6605-11.

18. Schmidt, M. D., and D. B. Oliver. 1989. SecA protein autogenously represses its own translation during normal protein secretion in Eschirichia coli. J. Bacteriol. 171(2) :643-9.

19. Slopek, S., B. Weber-Dacbrowska, M. Dacbrowski, and A. Kucharewicz-Krukowska. 1987. Results of bacteriophage treatment of suppurative bacterial infec-tions in the years 1981-1986. Arch. Immunol. Ther. Exp. (Warsz). 35:569-583.

20. Smith. H. W., and M. B. Huggins. 1982. Successful treatment of experimental Escherichia coli infections in mice using phage: its general superiority over antibiotics. J. Gen. Microbiol. 128:307-318.

21. Soothill, J. S. 1994. Bacteriophage prevents destruction of skin grafts by Pseudomonas aeruginosa. Burns. 20:209-211.

22. Soothill, J. S. 1992. Treatment of experimental infections of mice with bacterio phages. J. Med. Microbiol. 37:358-261.

23. Sternberg, N., and J. Coulby. 1987. Recognition of cleavage of the bacteriophage P1 packaging site (pac). II. Functional limits of pac and location of pac cleavage termini. J. Mol. Biol. 194(3):469-79.

24. Sullivan, S. M. 1994. Development of ribozymes for gene therapy. J. Invest Dermatol. 103(5 Supl):858-89S.

25. Taira, K., K. Nakagawa, S. Nishikawa, and K. Kurukawa. 1991. Construction of a noval RNA-transcript-trimming plasmid which can be used both in vitro and in place of run-off and (G)-free transcriptions and in vivo as multi-sequences transcription vectors. NAR. 19(9):5125-5130.

26. Tetart, F., and J. P. Bouche, 1992. Regulation of the expression of the cell-cycle gene ftsz by DicF antisense RNA. Division does not require a fixed number of FtsZ molecules. Mo. Microbiaol. 6(5):615-20.

27. Uhlenbeck, O. C. 1987. A small catalytic oligoribonucleotide. Nature. 328(6131):59

28. Weschler, J. A. and J. D. Gross. 1971. Escherichia coli mutants temperature sensitive for DNA systhesis. Mol. Gen. Genet. 113:273-284.

29. Yarmolinsky, M. B., and N. Sternberg. 1988. Bacteriophage, P1, p.291-438. In R. Calendar (ed.), The Bacteriophages, vol. 1, Plenum Press. New York.

30. Hawley-Nelson et al., Focus 15(3):73-83 (1992).

31. Felgner et al., Proc. Natl. Acad. Sci. U.S.A. 84:7413 (1987).

32. Stewart et al., Human Gene Therapy 3:267-275 (1992).

33. Nicolau et al., Methods Enzymol. 149:157 (1987).

What is claimed is:

1. A virion, modified to be non-replicative and comprising non-viral DNA.

2. The virion of claim 1, wherein the non-viral DNA encodes a ribozyme.

3. The virion of claim 2, wherein the non-viral DNA comprises a pathogen-specific promoter upstream from a sequence encoding a triple ribozyme comprising a) a 5′ autocatalytically cleaving ribozyme sequence, b) a catalytic ribozyme comprising a target RNA-specific binding site and c) a 3′ autocatalytically cleaving ribozyme sequence.

4. The virion of claim 3, wherein the non-viral DNA encodes more than one triple ribozyme.

5. A liposome comprising a nucleic acid comprising a pathogen-specific promoter upstream from a sequence encoding a triple ribozyme comprising a) a 5′ autocatalytically cleaving ribozyme sequence, b) a catalytic ribozyme comprising a target RNA-specific binding site and c) a 3′ autocatalytically cleaving ribozyme sequence.

6. The liposome of claim 5, wherein the nucleic acid encodes more than one triple ribozyme.

7. A nucleic acid comprising a pathogen-specific promoter upstream from a sequence encoding a triple ribozyme comprising a) a 5′ autocatalytically cleaving ribozyme sequence, b) a catalytic ribozyme comprising a target RNA-specific binding site and c) a 3′ autocatalytically cleaving ribozyme sequence.

8. The nucleic acid of claim 7, encoding more than one triple ribozyme.

9. The nucleic acid of claim 8, encoding at least two different triple ribozymes.

10. The nucleic acid of claim 8, encoding more than one copy of a triple ribozyme.

11. The nucleic acid of any of claims 7, 8, 9, 10, 12, 13 or 14, wherein at least one triple ribozyme is targeted to the rpoA transcript of the pathogen.

12. The nucleic acid of any of claims 7, 8, 9, 10, 11, 13 or 14 wherein at least one triple ribozyme is targeted to the seca transcript of the pathogen.

13. The nucleic acid of any of claims 7, 8, 9, 10, 11, 12 or 14, wherein at least one triple ribozyme is directed to the dnaG transcript of the pathogen.

14. The nucleic acid of any of claims 7, 8, 9, 10, 11, 12 or 13 wherein at least one triple ribozyme is directed to the ftsZ transcript of the pathogen.

15. A method of treating an infection in a subject, comprising administering to the subject the virion of any of claims 2, 3 or 4, whereby the ribozyme encoded by the non-phage DNA is expressed and the infectious agent is killed or weakened.

16. A method of treating an infection in a subject, comprising administering to the subject the liposome of claim 5 or 6, whereby the ribozyme encoded by the DNA is expressed and the infectious agent is killed or weakened.

17. A method of targeted delivery of a ribozyme to a pathogen in a subject, comprising
a) generating a virion of any of claims 2, 3 or 4; and
b) delivering the virion to the subject, whereby the pathogen-specific promoter directs transcription of the ribozyme in the cells of the pathogen.

18. The method of claim 17, wherein the virion is from bacteriophage lambda.

19. The method of claim 17, wherein the virion is from bacteriophage P1.

20. The method of claim 19, wherein the non-phage DNA further comprises a PAC site.

21. A method of targeted delivery of a ribozyme to a pathogen in a subject, comprising
a) generating a liposome of claim 5 or 6; and
b) delivering the liposome to the subject, whereby the pathogen-specific promoter directs transcription of the ribozyme in the cells of the pathogen.
